(19) United States
(12) Patent Application Publication
(10) Pub. No.: US 2024/0309036 A1
(43) Pub. Date: Sep. 19, 2024

(54) Phenol Compound-Linked Pentagalloyl Glucose Derivatives and Uses Thereof

(71) Applicant: GENTRIBIO INC., Gangwon-do (KR)

(72) Inventors: Chang Keun Yoon, Gangwon-do (KR); Hong Gyum Kim, Gangwon-do (KR); Myoung Ok Kim, Daegu (KR)

(73) Assignee: GENTRIBIO INC., Gangwon-do (KR)

(21) Appl. No.: 18/007,505

(22) PCT Filed: Oct. 24, 2022
(86) PCT No.: PCT/KR2022/016280

§ 371 Date: Dec. 1, 2022

(30) Foreign Application Priority Data
May 6, 2022 (KR) 10-2022-0055891

(51) Int. Cl. C07H 13/08; A61K 31/7024; A61P 3/10; A61P 35/00

(52) U.S. Cl. CPC C07H 13/08; A61K 31/7024; A61P 3/10; A61P 35/00

(57) ABSTRACT

Provided is a composition including a pentagalloyl glucose derivative and a phenol compound as active ingredients, the composition according to an aspect may not only be used for anti-cancer and anti-diabetic purposes without using additional additives, but also may be useful as an anti-diabetic drug for cancer patients, or an anti-cancer drug for diabetic patients.

### TECHNICAL FIELD

The present disclosure relates to phenol compound-linked pentagalloyl glucose derivatives and uses thereof.

### BACKGROUND ART

In general, galloyl glucoside is widely present in various plants exhibiting various biological and pharmacological activities, but it is known to be difficult to be isolated in large amounts due to its low free-form content, high polarity, and chemical properties of being easily oxidized. Among the galloyl glucosides, highly galloylated pentagalloyl glucose (PGG) is a compound in which five molecules of gallic acid and one molecule of glucose are combined in a form of an ester, which is present in some plants, such as plants belonging to the peony family, pomegranate, evening primrose, plants belonging to the sumac family, and green tea, and from a biological point of view, it is expected that galloyl glucosides with few galloyl groups will exhibit better pharmacological activity.

On the other hand, diabetes is increasing worldwide, and is also rapidly increasing in Korea, and 1 in 3 adults is included in a potential diabetes risk group. Although the number of cancer patients is very small compared to the diabetic population, cancer is the number one cause of death, and the mortality rate is high, and management of cancer patients occupies a large part of the health insurance budget.

For diabetic patients, the risk of pancreatic cancer, liver cancer, and uterine cancer is more than twice that of the normal group, and the incidence of colorectal cancer, bladder cancer, breast cancer, and non-Hodgkin's lymphoma is 20% to 50% higher than the normal group, and therefore, the risk of developing cancer and the risk of death due to cancer are shown to be high. The high risks are known to be associated with insulin resistance, hyperinsulinemia, insulin-like growth factors (IGF-1), and hyperglycemia.

Therefore, it has been reported that lowering blood sugar in diabetic patients can contribute to lowering the cancer risk by improving hyperglycemia and chronic inflammation. In particular, effectiveness of biguanide formulations, which are oral anti-hyperglycemic agents used for treating diabetes or prediabetes, has been reported. For example, metformin, which is inexpensive and easy to study with, has been studied to prevent malignant tumors (Korean Patent No. 10-2182946) or to show effects of amplifying effects of anti-cancer drugs. However, metformin frequently causes side effects such as nausea, vomiting, diarrhea, and appetite suppression, and cancer-causing substances are detected in some metformin products. Accordingly, there is a need to develop a therapeutic agent derived from a natural product with low toxicity that exhibits anti-diabetic effects in cancer patients or anti-cancer effects in diabetic patents.

Therefore, in order to solve the above issues, the present inventors confirmed that pentagalloyl glucose derivatives linked with phenol compounds may be effectively used in an anti-diabetic and anti-cancer treatment without an additional additive, and in particular, may be used as an effective therapeutic agent for complications, for example, as an anti-cancer agent for a diabetic patient, or an anti-diabetes agent for a cancer patient, and completed the present disclosure.

### DESCRIPTION OF EMBODIMENTS

### Technical Problem

An aspect is to provide a composition including a compound represented by Formula 1:

### Formula 1

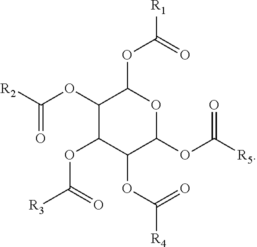

Another aspect is to provide a pharmaceutical composition for preventing or treating cancer, including the compound represented by Formula 1.

Still another aspect is to provide a pharmaceutical composition for preventing or treating diabetes, including the compound represented by Formula 1.

Still another aspect is to provide a pharmaceutical composition for preventing or treating diabetes in cancer patients, including the compound represented by Formula 1.

Still another aspect is to provide a pharmaceutical composition for preventing or treating cancer in diabetic patients, including the compound represented by Formula 1.

Still another aspect is to provide a method of preventing or treating cancer, including administering the compound represented by Formula 1 or a pharmaceutically acceptable salt thereof to a subject in need thereof.

Still another aspect is to provide a method of preventing or treating diabetes, including administering the compound represented by Formula 1 or a pharmaceutically acceptable salt thereof to a subject in need thereof.

Still another aspect is to provide a method of preventing or treating diabetes of cancer patients, including administering the compound represented by Formula 1 or a pharmaceutically acceptable salt thereof to a subject in need thereof.

Still another aspect is to provide a method of preventing or treating cancer of diabetic patients, including administering the compound represented by Formula 1 or a pharmaceutically acceptable salt thereof to a subject in need thereof.

### Solution to Problem

An aspect provides a composition including a compound represented by Formula 1:

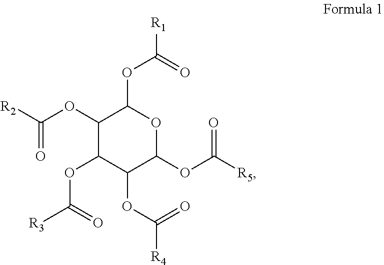

in Formula 1, R₁, R₂, R₃, R₄ or R5 is each independently a compound of Formula 2 or Formula 3 below.

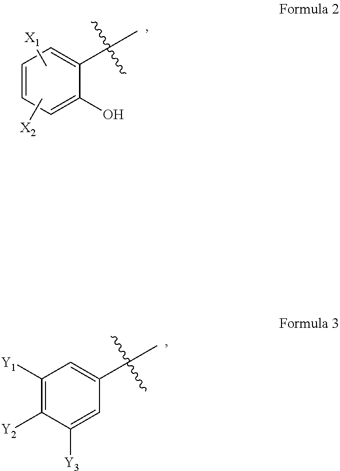

wherein at least one of R₁, R₂, R₃, R₄ or R₅ is a compound represented by Formula 2,
in Formula 2, X₁ or X₂ is each independently H, OH, a C₁ to C₁₀ alkyl that is substituted or unsubstituted, a C₂ to C₁₀ alkenyl that is substituted or unsubstituted, a C₂ to C₁₀ alkynyl that is substituted or unsubstituted, or a C₁ to C₁₀ alkoxy that is substituted or unsubstituted, and
in Formula 3, Y₁, Y₂, or Y₃ is each independently H, OH, a C₁ to C₁₀ alkyl that is substituted or unsubstituted, a C₂ to C₁₀ alkenyl that is substituted or unsubstituted, a C₂ to C₁₀ alkynyl that is substituted or unsubstituted, or a C₁ to C₁₀ alkoxy that is substituted or unsubstituted.

In an embodiment, in Formula 2, X₁ or X₂ may each independently be H, OH, or a C₁ to C₁₀ alkyl that is substituted or unsubstituted.

In an embodiment, in Formula 3, Y₁ or Y₂ may each independently be H, OH, or a C₁ to C₁₀ alkyl that is substituted or unsubstituted.

The term “substituted”, used herein, refers to being substituted with one or more substituents, for example, a hydroxy group, an alkyl group, an alkoxy group, a mercapto group, a cycloalkyl group, a substituted cycloalkyl group, a heterocyclic group, a substituted heterocyclic group, an aryl group, a substituted aryl group, a heteroaryl group, a substituted heteroaryl group, an aryloxy group, a substituted aryloxy group, a halogen group, a trifluoromethyl group, a cyano group, a nitro group, an oxo group, an amino group, an amido group, an aldehyde group, an acyl group, an oxyacyl group, a carboxyl group, a carbamate group, a sulfonyl group, a sulfonamide group, a sulfuryl group, etc.

The term “alkyl”, used herein, refers to a saturated aliphatic hydrocarbon group containing 1 to 10, for example, 1 to 8, 1 to 6, or 1 to 4 carbon atoms, the alkyl group being a straight chain or a branched chain. In addition, the alkyl includes cycloalkyl, heteroalkyl, and heterocycloalkyl. Examples include, but are not limited to, methyl, ethyl, propyl, isopropyl, butyl, isobutyl, sec-butyl, tert-butyl, n-pentyl, n-heptyl, or 2-ethylhexyl.

The alkyl may be substituted by at least one substituent, for example, a halo-, phospho-, or cyclo-aliphatic group (for example, cycloalkyl or cycloalkenyl), a heterocycloaliphatic group (for example, heterocycloalkyl or heterocycloalkenyl), aryl, heteroaryl, alkoxy, aroyl, heteroaroyl, acyl (for example, (aliphatic)carbonyl, (cycloaliphatic)carbonyl, or (heterocycloaliphatic)carbonyl), nitro, cyano, amido (for example, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino alkylaminocarbonyl, cycloalkylaminocarbonyl, heterocycloalkylaminocarbonyl, arylaminocarbonyl, or heteroarylaminocarbonyl), amino (for example, aliphatic amino, cycloaliphatic amino, or heterocycloaliphaticamino), sulfonyl (for example, aliphatic-SO₂—), sulfinyl, sulfanyl, sulfoxy, urea, thiourea, sulfamoyl, sulfamide, oxo, carboxy, carbamoyl, cycloaliphaticoxy, heterocycloaliphaticoxy, aryloxy, heteroaryloxy, aralkyloxy, heteroarylalkoxy, alkoxycarbonyl, alkylcarbonyloxy, or hydroxy. A substituted alkyl may be carboxyalkyl (for example, HOOC-alkyl, alkoxycarbonylalkyl, or alkylcarbonyloxyalkyl), cyanoalkyl, hydroxyalkyl, alkoxyalkyl, acylalkyl, aralkyl, (alkoxyaryl)alkyl, (sulfonylamino)alkyl (for example, (alkyl-SO₂-amino)alkyl), aminoalkyl, amidoalkyl, (cycloaliphatic)alkyl, haloalkyl, amine cationx, quaternary ammonium, or quaternary phosphonium.

The term “heteroalkyl”, used herein, refers to alkyl consisting of carbon atoms, and one or more heteroatoms selected from the group consisting of O, N, and S. The nitrogen and sulfur atoms may be arbitrarily oxidized and the heteroatoms O, N and/or S may be placed at any internal position of the heteroalkyl group.

The term “alkenyl”, used herein, refers to an aliphatic carbon group containing 2 to 10, for example, 2 to 8, 2 to 6, or 2 to 4 carbon atoms and at least one double bond. Similar to an alkyl group, an alkenyl group may be a straight chain or a branched chain. In addition, the alkenyl includes cycloalkenyl, heteroalkenyl, and cycloheteroalkenyl. The alkenyl may be, for example, allyl, isoprenyl, 2-butenyl, or 2-hexenyl, but is not limited thereto.

The alkenyl may be substituted with at least one substituent, for example, a halo-, phospho-, or cyclo-aliphatic group (for example, cycloalkyl or cycloalkenyl), a heterocycloaliphatic group (for example, heterocycloalkyl or heterocycloalkenyl), aryl, heteroaryl, alkoxy, aroyl, heteroaroyl, acyl (for example, (aliphatic)carbonyl, (cycloaliphatic)carbonyl, or (heterocycloaliphatic)carbonyl), nitro, cyano, amido (for example, (cycloalkylalkyl)carbonylamino, arylcarbonylamino, aralkylcarbonylamino, (heterocycloalkyl)carbonylamino, heteroarylcarbonylamino, (heterocycloalkylalkyl)carbonylamino, heteroaralkylcarbonylamino alkylaminocarbonyl, cycloalkylaminocarbonyl, heterocycloalkylaminocarbonyl, arylaminocarbonyl, or heteroarylaminocarbonyl), amino (for example, aliphatic amino, cycloaliphatic amino, or heterocycloaliphaticamino), sulfonyl (for example, aliphatic-SO₂—), sulfinyl, sulfanyl, sulfoxy, urea, thiourea, sulfamoyl, sulfamide, oxo, carboxy, carbamoyl, cycloaliphaticoxy, heterocycloaliphaticoxy, aryloxy, heteroaryloxy, aralkyloxy, heteroarylalkoxy, alkoxycarbonyl, alkylcarbonyloxy, or hydroxy. Substituted alkenyl may be cyanoalkenyl, alkoxyalkenyl, acylalkenyl, hydroxyalkenyl, aralkenyl, (alkoxyaryl)alkenyl, (sulfonylamino)alkenyl (for example, (alkyl-SO₂—amino)al kenyl), aminoalkenyl, amidoalkenyl, (cycloaliphatic)alkenyl, or haloalkenyl.

The term “heteroalkenyl”, used herein, refers to alkenyl consisting of carbon atoms and one or more heteroatoms selected from the group consisting of O, N, and S. The nitrogen and sulfur atoms may be arbitrarily oxidized, and the heteroatoms O, N and/or S may be placed at any internal position of the heteroalkenyl group.

The term “alkynyl”, used herein, refers to a aliphatic hydrocarbon group containing 2 to 10, for example, 2 to 8, 2 to 6, or 2 to 4 carbon atoms, and having at least 1 triple bond, and the alkynyl group may be a straight chain or a branched chain. The alkynyl includes cycloalkynyl, heteroalkynyl, and cycloheteroalkynyl. For example, the alkynyl group may be propargyl or butynyl.

The alkynyl may be arbitrarily substituted by at least one substituent, for example, aroyl, heteroaroyl, alkoxy, cycloalkyloxy, heterocycloalkyloxy, aryloxy, heteroaryloxy, aralkyloxy, nitro, carboxy, cyano, halo, hydro oxy, sulfo, mercapto, sulfanyl (for example, aliphaticsulfanyl or cycloaliphaticsulfanyl), sulfinyl (for example, aliphaticsulfinyl or cycloaliphaticsulfinyl), sulfonyl (for example, aliphatic-SO₂—, aliphaticamino-SO₂—, or cycloaliphatic-SO₂—), amido (for example, aminocarbonyl, alkylaminocarbonyl, alkylcarbonylamino, cycloalkylaminocarbonyl, heterocycloalkylaminocarbonyl, cycloalkylcarbonylamino, arylaminocarbonyl, arylcarbonylamino, aralkylcarbonylamino, (heterocycloalkyl)carbonylamino, (cycloalkylalkyl)carbonylamino, heteroaralkylcarbonylamino, heteroaryl carbonylamino, or heteroarylaminocarbonyl), urea, thiourea, sulfamoyl, sulfamide, alkoxycarbonyl, alkylcarbonyloxy, a cycloaliphatic group, a heterocycloaliphatic group, aryl, heteroaryl, acyl (for example, (cycloaliphatic)carbonyl, or (heterocycloaliphatic)carbonyl), amino (for example, aliphatic amino), sulfoxy, oxo, carboxy, carbamoyl, (cycloaliphatic)oxy, (heterocycloaliphatic)oxy, or (heteroaryl)alkoxy.

The term “heteroalkynyl”, used herein, refers to alkynyl consisting of carbon atoms and one or more heteroatoms selected from the group consisting of O, N, and S. The nitrogen and sulfur atoms may be arbitrarily oxidized, and the heteroatoms O, N and/or S may be placed at any internal position of the heteroalkynyl group.

The term “alkoxy”, used herein, refers to a group in which a hydrogen atom of hydroxyl is substituted with the alkyl, alkenyl or alkynyl. The alkoxy may be C₁ to C₁₀ alkoxy, such as methoxy, ethoxy, propoxy, butoxy, and pentoxy.

The term “aryl”, used herein, refers to an aromatic ring system including 6 to 16 ring atoms, and includes heteroaryl. The aryl group may be a monocyclic, bicyclic or tricyclic group, or may be linked by a bond to form a biaryl group. Typically, the aryl group may be phenyl, naphthyl, or biphenyl. In an embodiment, the aryl group may include an alkylene linking group to form an arylalkyl group, for example, a benzyl group. The aryl group may have 6 to 12 ring members such as phenyl, naphthyl, or biphenyl, and the aryl may be substituted or unsubstituted.

The term “heteroaryl”, used herein, refers to a monocyclic, fused bicyclic, or tricyclic aromatic ring assembly, including 5 to 16 ring atoms, wherein 1 to 5 of the atoms constituting the ring may be heteroatoms such as N, O or S. The heteroatoms may be oxidized and include, for example, N-oxides, —S(O)—, and —S(O)₂—, but are not limited thereto. A heteroaryl group may include any number of ring atoms, for example, 3 to 6, 4 to 6, 5 to 6, 3 to 8, 4 to 8, 5 to 8, 6 to 8, 3 to 9, 3 to 10, 3 to 11, or 3 to 12 ring members. According to an embodiment, a heteroaryl group has 5 to 8 ring members and 1 to 4 heteroatoms, or 5 to 8 ring members and 1 to 3 heteroatoms, or 5 to 6 ring members and 1 to 4 heteroatoms. heteroatoms, or 5 to 6 ring members and 1 to 3 heteroatoms. The heteroaryl group may include a group such as a pyrrole, pyridine, imidazole, pyrazole, triazole, tetrazole, pyrazine, pyrimidine, pyridazine, triazine (1,2,3-, 1,2,4-, and 1,3; 5-isomer), thiophene, furan, thiazole, isothiazole, oxazole, and isoxazole group. The heteroaryl group may also include an aromatic ring system, for example, benzopyrrole fused to a phenyl ring, such as indole and isoindole, benzopyridine such as quinoline and isoquinoline, benzopyrazine, benzopyrimidine, benzopyridazine such as phthalazine and cinnoline, benzothiophene, and benzofuran. In addition, the heteroaryl group may include a heteroaryl ring linked by a bond, for example, bipyridine, and the heteroaryl group may be substituted or unsubstituted.

The heteroaryl group may be linked by any position on the ring. For example, pyrroles may include 1-, 2-, and 3-pyrroles; pyridines may include 2-, 3-, and 4-pyridines; imidazoles may include 1-, 2-, 4-, and 5-imidazoles; pyrazoles may include 1-, 3-, 4-, and 5-pyrazoles; triazoles may include 1-, 4-, and 5-triazoles; tetrazoles may include 1-, and 5-tetrazoles; pyrimidines may include 2-, 4-, 5-, and 6-pyrimidines; pyridazines may include 3- and 4-pyridazines; 1,2,3-triazines may include 4- and 5-triazines; 1,2,4-triazines may include 3-, 5-, and 6-triazines; 1,3,5-triazines may include 2-triazine; thiophenes may include 2- and 3-thiophenes; furans may include 2- and 3-furans; thiazoles may include 2-, 4-, and 5-thiazoles; isothiazoles may include 3-, 4-, and 5-isothiazoles; oxazoles may include 2-, 4-, and 5-oxazoles; isoxazoles may include 3-, 4-, and 5-isoxazoles; indoles may include 1-, 2-, and 3-indoles; isoindoles may include 1- and 2-isoindoles; quinolines may include 2-, 3-, and 4-quinolines; Isoquinolines may include 1-, 3-, and 4-isoquinolines; quinazolines may include 2- and 4-quinazolines; cinnolines may include 3- and 4-cinnolines; benzothiophenes may include 2- and 3-benzothiophenes; and benzofurans may include 2- and 3-benzofurans.

In an embodiment, the compound of Formula 2 may be a compound represented by the following Formula 4, but is not limited thereto:

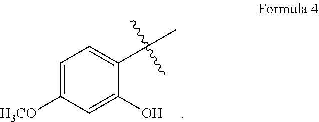 [SMILES: *c1ccc(OC)cc1O ; "."]

In an embodiment, the compound of Formula 3 may be a compound represented by the following Formula 5, but is not limited thereto:

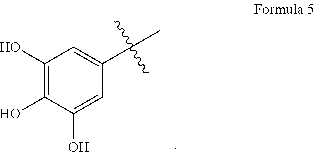 [SMILES: *c1cc(O)c(O)c(O)c1]

The term “pharmaceutically acceptable”, used herein, means being physiologically acceptable and not normally causing allergic reactions such as gastrointestinal disorders and dizziness, or similar reactions when administered to humans.

The term “pharmaceutically acceptable salt”, used herein, refers to a salt according to an aspect of the present disclosure that is pharmaceutically acceptable and has a desired pharmacological activity of the parent compound. Salts of a parent compound may be synthesized from the parent compound containing a basic or acidic moiety by a chemical method in the art. Generally, such salts may be prepared by reacting the free acid form of these compounds with a stoichiometric amount of an appropriate base, for example, sodium, calcium, magnesium, or potassium, or by reacting the free base form of these compounds with a stoichiometric amount of an appropriate acid. These reactions are typically carried out in water or in organic solvents or a mixture of the two. In general, where practicable, non-aqueous media such as ether, ethyl acetate, ethanol, isopropanol, or acetonitrile may be used. The pharmaceutically acceptable salt includes both an addition salt of an acid or a base and a stereochemically isomeric form thereof, and may be, for example, an addition salt of an organic acid or an inorganic acid. The salt is not particularly limited, within the range that the salt maintains the activity of the parent compound and does not cause an undesirable effect in the subject to be administered.

Such salts include inorganic and organic salts, for example, acetic acid, nitric acid, aspartic acid, sulfonic acid, sulfuric acid, maleic acid, glutamic acid, formic acid, succinic acid, phosphoric acid, phthalic acid, tannic acid, tartaric acid, hydrobromic acid, propionic acid, benzenesulfonic acid, benzoic acid, stearic acid, lactic acid, bicarboxylic acid, bisulfuric acid, bitartaric acid, oxalic acid, butyric acid, calcium idet, carbonic acid, chlorobenzoic acid, citric acid, idetic acid, toluene sulfonic acid, fumaric acid, gluceptic acid, ethylic acid, pamoic acid, gluconic acid, methyl nitric acid, malonic acid, hydrochloric acid, hydroiodoic acid, hydroxynaphtolic acid, isethionic acid, lactobionic acid, mandelic acid, mucic acid, nafsilic acid, muconic acid, p-nitromethanesulfonic acid, hexamic acid, pantothenic acid, monohydrogen phosphoric acid, dihydrogen phosphoric acid, salicylic acid, sulfamic acid, sulfanilic acid, and methanesulfonic acid.

In addition, the form of the salt includes salts of alkali and alkaline earth metals such as ammonium salts, lithium salts, sodium salts, potassium salts, magnesium salts, and calcium salts, for example, salts with an organic base such as benzathine, N-methyl-D-glucamine, and hydrabamine salts, hydride salts with organic bases such as lavamine salts, and salts with an amino acid such as arginine and lysine. In addition, the salt form may also be converted to the free form by treatment with a suitable base or acid.

The compound represented by Formula 1 may be one obtained by linking a pentagalloyl glucose derivative or a pharmaceutically acceptable salt thereof; and a phenol compound or a derivative thereof.

The pentagalloyl glucose derivative may be a compound shown in Table 1 below.

TABLE 1

| Formula | Structure |
|---|---|
| 6 | 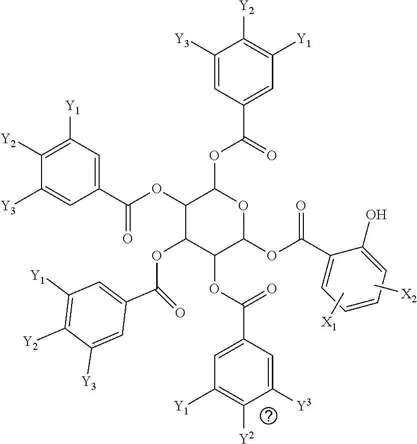 |
| 7 | 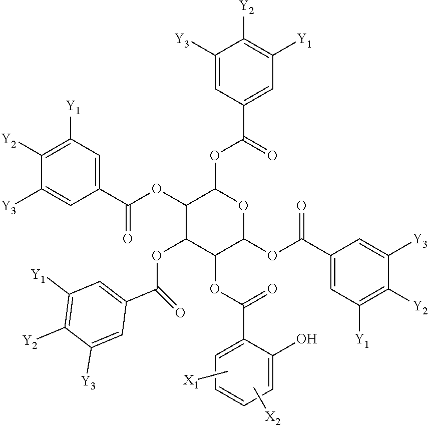 |
| 8 | 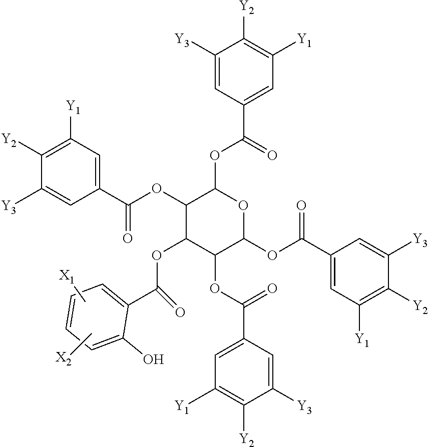 |
| 9 | 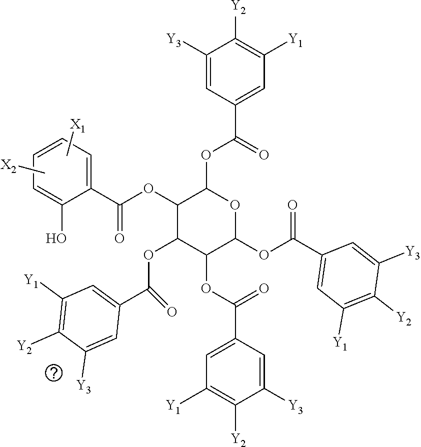 |
| 10 | 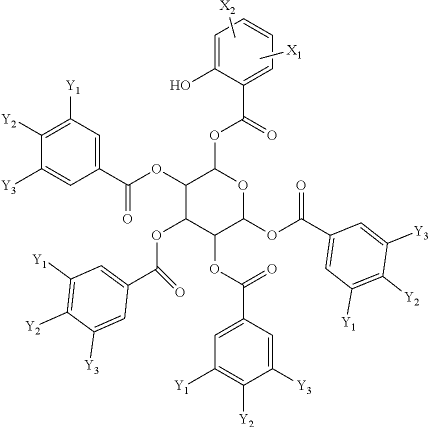 |
| 11 | 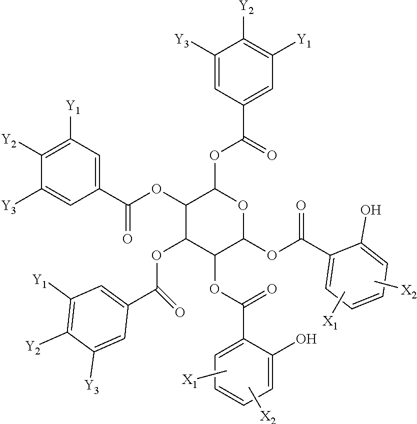 |
| 12 | 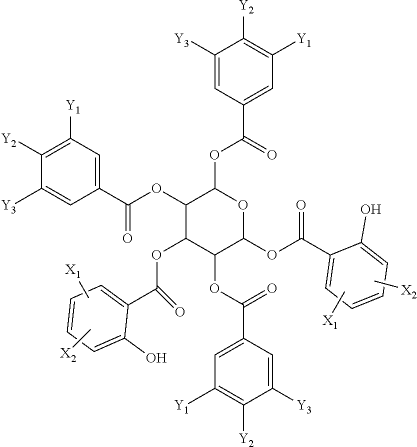 |
| 13 | 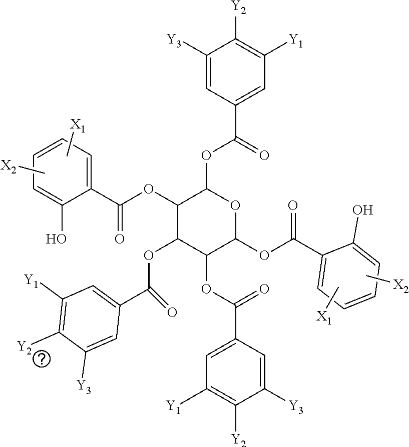 |
| 14 | 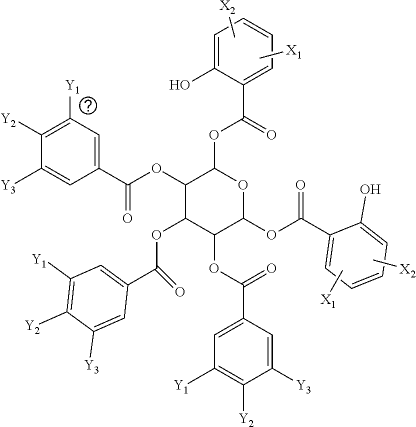 |
| 15 | 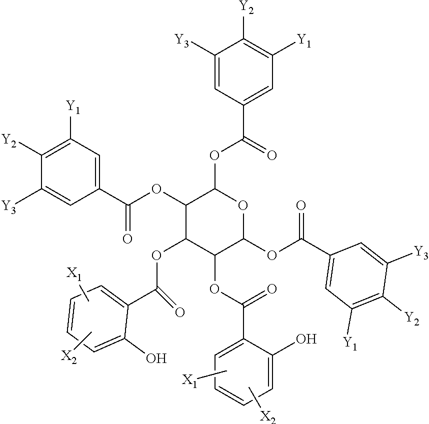 |
| 16 | 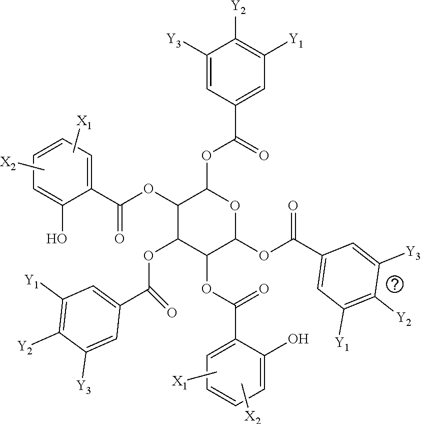 |
| 17 | 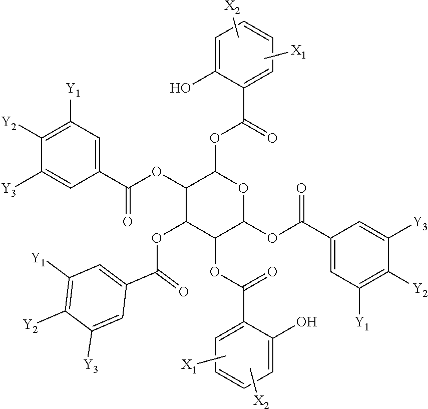 |
| 18 | 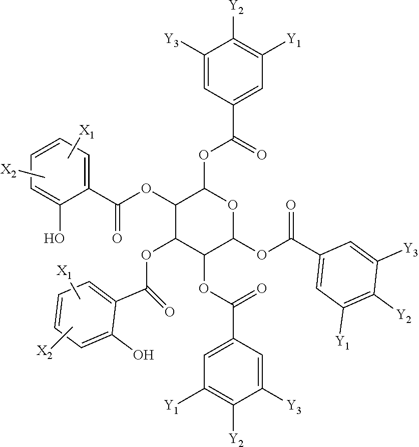 |
| 19 | 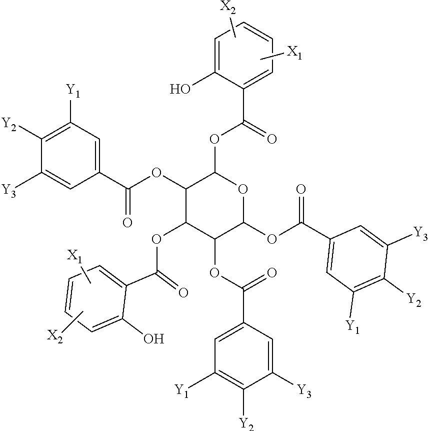 |
| 20 | 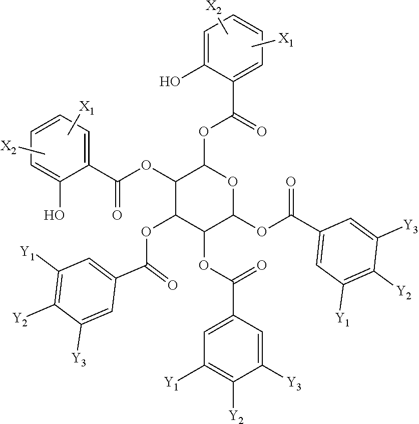 |
| 21 | 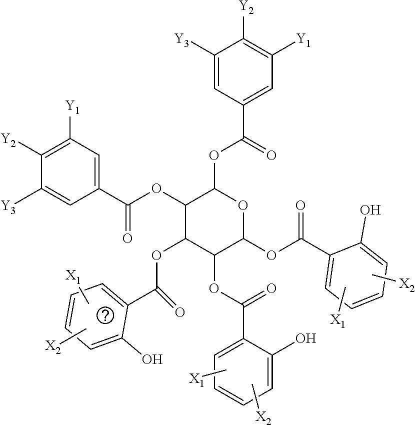 |
| 22 | 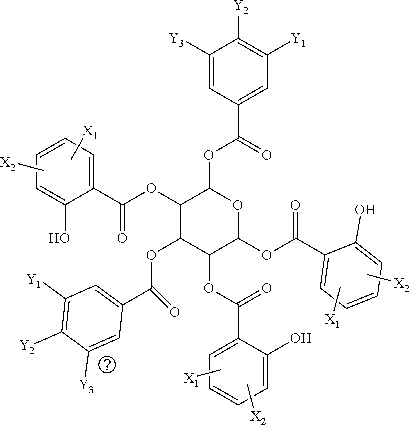 |
| 23 | 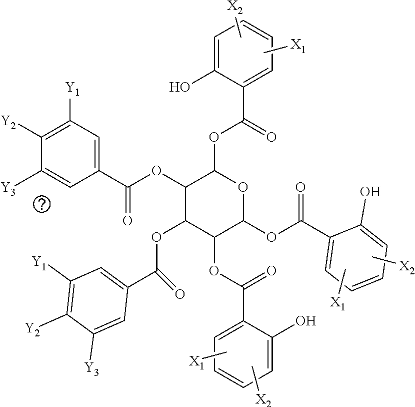 |
| 24 | 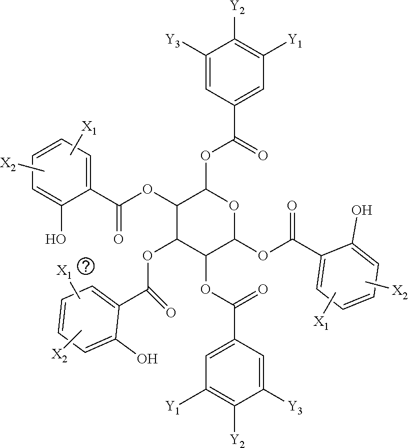 |
| 25 | 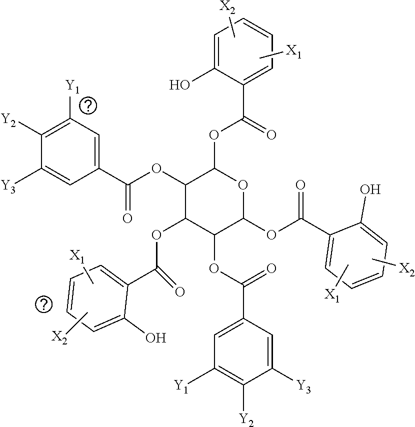 |
| 26 | 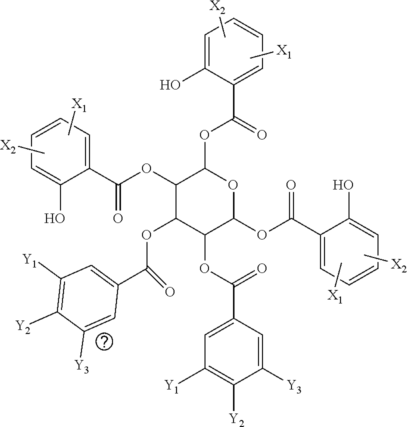 |
| 27 | 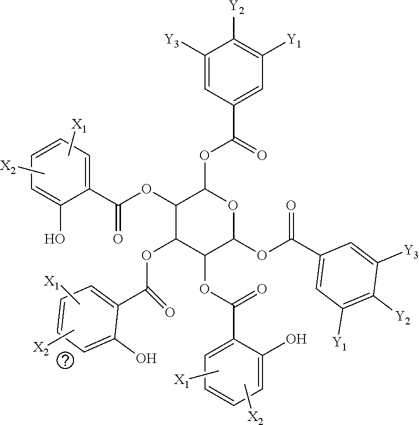 |
| 28 | 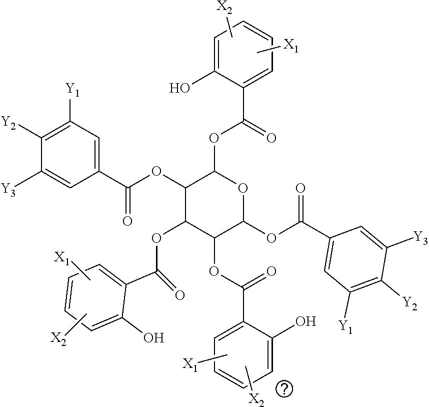 |
| 29 | 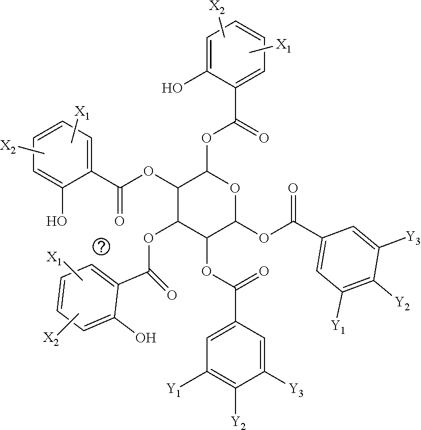 |
| 30 | 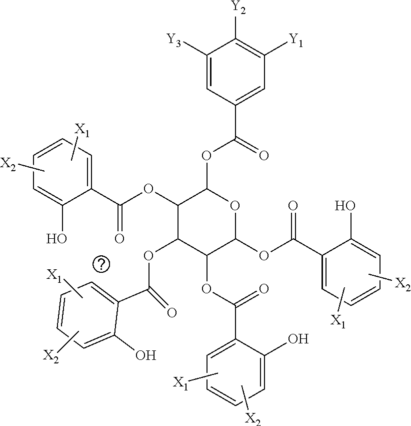 |
| 31 | 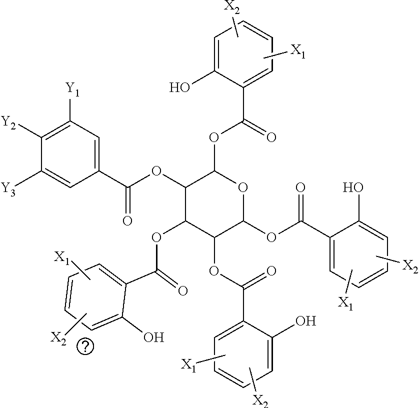 |
| 32 | 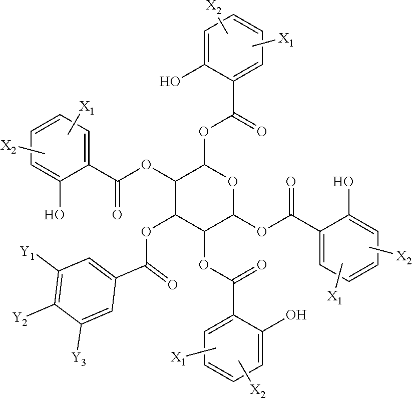 |
| 33 | 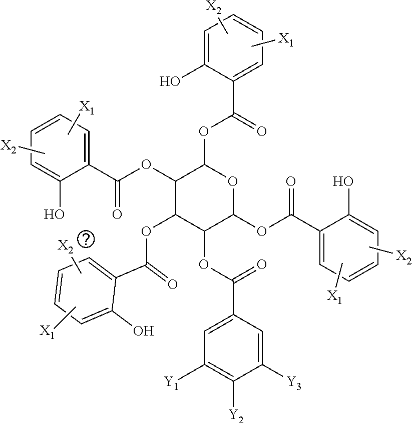 |
| 34 | 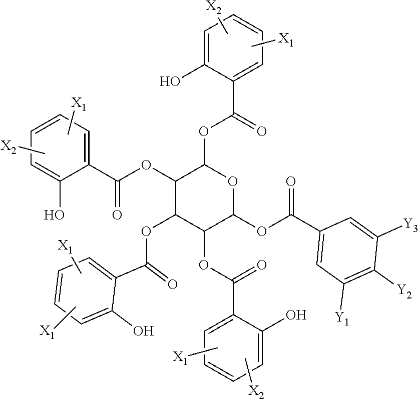 |
| 35 | 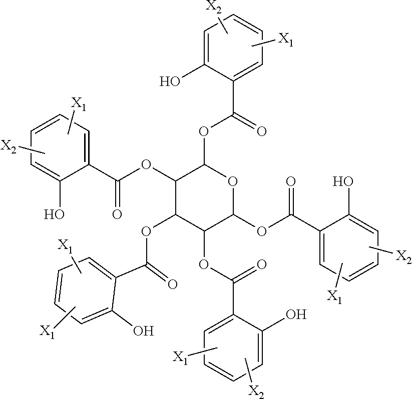 |

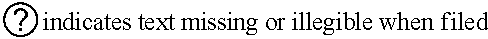

In the compounds shown in Table 1, X₁ or X₂ is each independently H, OH, a C₁ to C₁₀ alkyl that is substituted or unsubstituted, a C₂ to C₁₀ alkenyl that is substituted or unsubstituted, a C₂ to C₁₀ alkynyl that is substituted or unsubstituted, or a C₁ to C₁₀ alkoxy that is substituted or unsubstituted; and Y₁, Y₂ or Y₃ are each independently H, OH, a C₁ to C₁₀ alkyl that is substituted or unsubstituted, a C₂ to C₁₀ alkenyl that is substituted or unsubstituted, a C₂ to C₁₀ alkynyl that is substituted or unsubstituted, or a C₁ to C₁₀ alkoxy that is substituted or unsubstituted.

The linking of the compounds shown in [Table 1] may be linking a pentagalloyl glucose derivative or a pharmaceutically acceptable salt thereof; and a phenol compound or a derivative thereof in the presence of a base and a solvent for 5 hours to 20 hours, for example, 10 hours to 20 hours, 10 hours to 15 hours, or 10 hours to 13 hours. The reaction may be carried out at room temperature, and a person skilled in the art may appropriately change the reaction time depending on the temperature.

The base is NaH, Na₂SO₄, lithium diisopropylamide (LDA), 4-dimethylaminopyridine (DMAP), triethylamine (TEA), pyridine, ammonia, methylamine, ethylamine, propylamine, isopropylamine, dimethylamine, diethylamine, dipropylamine, diisopropylamine, trimethylamine, tripropylamine, triisopropylamine, aniline, methylaniline, dimethylaniline, pyridine, azazurolidine, benzylamine, methylbenzylamine, dimethylbenzylamine, 2,6-lutidine, morpholine, piperidine, piperazine, proton-sponge, ammonium hydroxide, triethanolamine, ethanolamine, or Trizma.

The solvent may be dimethylacetamide (DMAc), dichloromethane (DCM), tetrahydrofuran (THF), dimethylformamide (DMF), acetonitrile (ACN), DMAP, water, acetic acid, acetone, dioxane, benzene, 1-butanol, 2-butanol, tert-butyl alcohol, carbon tetrachloride, chloroform, cyclohexane, hexane, diethyl ether, dimethyl sulfoxide (DMSO), ethanol, ethyl acetate, ethylene glycol, glycerin, heptane, pentane, pyridine, toluene, hydrochloric acid, and triethyl amine.

Another aspect provides a pharmaceutical composition for preventing or treating cancer including the compound represented by Formula 1 or a pharmaceutically acceptable salt thereof.

Still another aspect provides a pharmaceutical composition for preventing or treating diabetes including a compound represented by Formula 1 or a pharmaceutically acceptable salt thereof.

Still another aspect provides a method of preventing or treating cancer, including administering a compound represented by Formula 1 or a pharmaceutically acceptable salt thereof to a subject in need thereof.

Still another aspect provides a method of preventing or treating diabetes, including administering a compound represented by Formula 1 or a pharmaceutically acceptable salt thereof to a subject in need thereof:

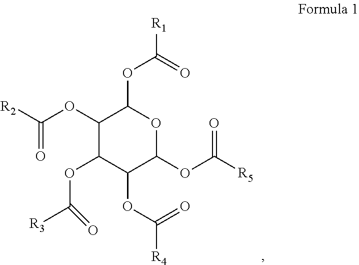

in Formula 1, R₁, R₂, R₃, R₄ or R₅ is each independently a compound of Formula 2 or 3:

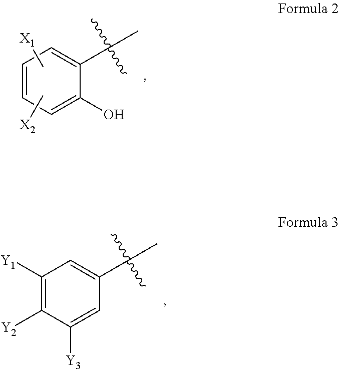

at least one of R₁, R₂, R₃, R₄ and R₅ is a compound represented by Formula 2,
in Formula 2, X₁ or X₂ is each independently H, OH, a C₁ to C₁₀ alkyl that is substituted or unsubstituted, a C₂ to C₁₀ alkenyl that is substituted or unsubstituted, a C₂ to C₁₀ alkynyl that is substituted or unsubstituted, or a C₁ to C₁₀ alkoxy that is substituted or unsubstituted, and
in Formula 3, Y₁, Y₂, or Y₃ is each independently H, OH, a C₁ to C₁₀ alkyl that is substituted or unsubstituted, a C₂ to C₁₀ alkenyl that is substituted or unsubstituted, a C₂ to C₁₀ alkynyl that is substituted or unsubstituted, or a C₁ to C₁₀ alkoxy that is substituted or unsubstituted.

The compound represented by Formula 1 is as described above.

The term “prevention”, used herein, collectively refers to partially or completely delaying or preventing an onset or recurrence of a disease, disorder, or ancillary symptoms thereof, preventing an acquisition or reacquisition of a disease or disorder, or reducing a risk of an acquisition of a disease or disorder. The prevention refers to any action of suppressing or delaying an occurrence of inflammation or inflammation-related diseases, disorders, or symptoms by administering the composition according to the present disclosure.

The term “treatment”, used herein, refers to any action by which a disease, disorder, or an ancillary symptom thereof is improved or beneficially changed.

According to an embodiment, the cancer may be a blood cancer or a solid cancer.

The blood cancer may be selected from the group consisting of acute myeloid leukemia, acute lymphoblastic leukemia, chronic myelogenous leukemia, multiple myeloma, and lymphoma, but is not limited thereto.

The solid cancer may be selected from the group consisting of breast cancer, colorectal cancer, head and neck cancer, lung cancer, stomach cancer, skin cancer, colon cancer, prostate cancer, bladder cancer, kidney cancer, rectal cancer, thyroid cancer, liver cancer, cervical cancer, skin cancer, anal cancer, urethral cancer, ovarian cancer, esophageal cancer, and pancreatic cancer.

The colorectal cancer includes malignant tumors occurring in one or more sites selected from the group consisting of ascending colon, transverse colon, descending colon, sigmoid colon, and rectal mucosa. In addition, the colorectal cancer may be one or more types selected from the group consisting of adenocarcinoma, lymphoma, malignant carcinoid tumor, leiomyosarcoma, Kaposi's sarcoma, and squamous cell carcinoma, but is not limited thereto.

In addition, the breast cancer may be estrogen receptor-positive breast cancer, estrogen receptor-positive/HER2-negative breast cancer, HER2-negative breast cancer, HER2-positive breast cancer, estrogen receptor-negative breast cancer, progesterone receptor-positive breast cancer, or progesterone receptor-negative breast cancer.

The pancreatic cancer may be a mucinous cystic tumor, intraductal papillary mucinous neoplasm, solid pseudopapillary tumor, a lymphoidepithelial cyst, pancreatic ductal adenocarcinoma, pancreatic acinar cell carcinoma, or pancreatic neuroendocrine tumor.

In an embodiment, the composition may further include an antioxidant, wherein the antioxidant may be at least one selected from the group consisting of nicotinamide, anthocyanin, benzenediol abiethane diterpene, carnosine, carotenoids, xanthophylls, and carotenoids in saffron, curcuminoids, cyclopentenone prostaglandins, flavonoids, prenylflavonoids, retinoids, stilbenoids, uric acid, vitamin A, vitamin B1, vitamin B2, vitamin B3, vitamin B6, vitamin B9, vitamin B12, vitamin C, vitamin E, selenium, zinc, inhibitors and scavengers of lipid peroxidation and by-products thereof, tirilazad, and analogs, derivatives, salts, and combinations thereof.

In addition, the composition may further include a pharmaceutically effective amount of a physiologically active ingredient or may include one or more pharmaceutically acceptable carriers, excipients, or diluents. The pharmaceutically effective amount means an amount sufficient to exhibit a desired physiological or pharmacological activity when the physiologically active ingredient is administered to an animal or human. The pharmaceutically effective amount may be appropriately changed according to the age, weight, health condition, sex, administration route, and treatment period of the subject to be administered.

The “physiologically active ingredient” refers to a substance capable of inducing a desired biological or pharmacological effect by promoting or inhibiting physiological functions in the body of an animal or human, and is a chemical or biological substance or compound suitable for administrating to an animal or human, and may (1) have a preventive effect in an organism by preventing unwanted biological effects, such as infection, (2) alleviate conditions resulting from a disease, for example, alleviate pain or infection resulting from a disease, and (3) play a role in alleviating, reducing, or completely eliminating a disease from an organism. In addition, “the physiologically active ingredient” may be used interchangeably with the term “therapeutic agent”.

The physiologically active ingredient may be selected from the group consisting of proteins, anti-cancer drugs, anti-inflammatory drugs, anti-biotics, anti-bacterial drugs, hormones, genes, and vaccines, but is not limited thereto.

The pharmaceutical composition may be formulated by using a diluent or excipient such as a filler, an extender, a binder, a wetting agent, a disintegrant, and a surfactant by a method known in the art, and the formulation may be an oral or parenteral dosage form. The oral dosage form may be granules, powders, liquids, tablets, capsules, dry syrups, or a combination thereof, and the parenteral dosage form may be injections, but is not limited thereto.

The administration may be by a method known in the art. The administration may be administering directly to a subject by any means, for example, intravenous, intramuscular, oral, transdermal, mucosal, intranasal, intratracheal, or subcutaneous administration. The administration may be systemic or local.

The subject may include, but is not limited to, mammals such as humans, cattle, horses, pigs, dogs, sheep, goats, or cats. The subject may be a subject in need of an effect of improving conditions related to cancer and diabetes.

A therapeutically effective dose of the pharmaceutical composition may be administered in an amount of 0.01 mg to 1000 mg, for example, 0.1 mg to 500 mg, or 0.1 mg to 300 mg per 1 kg of body weight per day, or may be administered several times in aliquots. However, the therapeutically effective dosage or therapeutically effective amount may vary depending on the formulation method, administration method, administration time and/or route of administration of the pharmaceutical composition. In addition, the therapeutically effective dosage or therapeutically effective amount may vary depending on several factors such as a type and degree of response to be achieved by the administration of the composition, a type, age, weight, general health condition, symptoms or degree of disease, sex, diet, and excretion of the subject to be administered, drugs and other compositions used together simultaneously, or at different times, and similar factors well known in the pharmaceutical field, and those of ordinary skill in the art may easily determine and prescribe an effective dosage for the desired treatment.

Still another aspect provides a pharmaceutical composition for preventing or treating diabetes of cancer patients including a compound represented by Formula 1 or a pharmaceutically acceptable salt thereof.

Still another aspect provides a pharmaceutical composition for preventing or treating cancer of diabetic patients including a compound represented by Formula 1 or a pharmaceutically acceptable salt thereof.

Still another aspect is to provide a method of preventing or treating diabetes of cancer patients, including administering the compound represented by Formula 1 or a pharmaceutically acceptable salt thereof to a subject in need thereof.

Still another aspect is to provide a method of preventing or treating cancer of diabetic patients, including administering the compound represented by Formula 1 or a pharmaceutically acceptable salt thereof to a subject in need thereof:

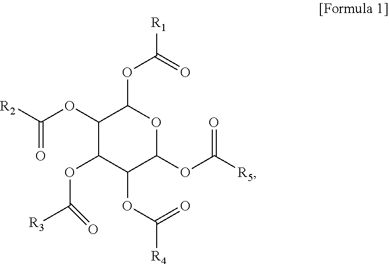

in Formula 1, R₁, R₂, R₃, R₄ or R₅ is each independently a compound of Formula 2 or 3:

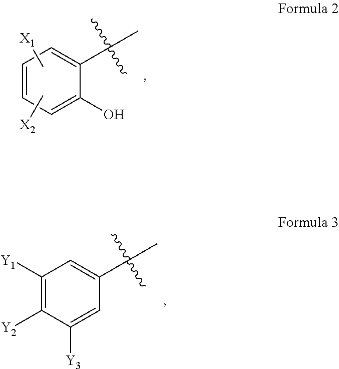

at least one of R₁, R₂, R₃, R₄ and R₅ is a compound represented by Formula 2,
X₁ or X₂ is each independently H, OH, a C₁ to C₁₀ alkyl that is substituted or unsubstituted, a C₂ to C₁₀ alkenyl that is substituted or unsubstituted, a C₂ to C₁₀ alkynyl that is substituted or unsubstituted, or a C₁ to C₁₀ alkoxy that is substituted or unsubstituted, and
Y₁, Y₂, or Y₃ is each independently H, OH, a C₁ to C₁₀ alkyl that is substituted or unsubstituted, a C₂ to C₁₀ alkenyl that is substituted or unsubstituted, a C₂ to C₁₀ alkynyl that is substituted or unsubstituted, or a C₁ to C₁₀ alkoxy that is substituted or unsubstituted.

The compound may have an effect of preventing or treating cancer in diabetic patients and/or preventing or treating diabetes in cancer patients by inhibiting onset of cancer in a diabetic patient and/or diabetes in a cancer patient. Advantageous Effects of Disclosure

The composition including a pentagalloyl glucose derivative and a phenol compound as active ingredients according to an aspect, has an effect of being useful as a drug for anti-diabetic treatment of cancer patients or anti-cancer treatment of diabetic patients without using additional additives.

### BRIEF DESCRIPTION OF DRAWINGS

FIG. 1 shows NMR results of (2R,3R,4S,5R,6R)-2-((2-(benzyloxy)-4-methoxybenzoyl)oxy)-6-(((3,4,5-tris(benzyloxy))benzoyl)oxy)methyl)tetrahydro-2H-pyran-3,4,5-triyltris(3,4,5-tris(benzyloxy)benzoate) (Formula 38); FIG. 1A shows ¹H NMR results, and FIG. 1B shows ¹³C NMR results.

FIG. 2 shows NMR results of a phenol compound-linked pentagalloyl glucose derivative according to an aspect (GBD-CP1-1.0) (Formula 39); FIG. 2A shows ¹H NMR results, and FIG. 2B shows ¹³C NMR results.

FIG. 3 shows HPLC and HRMS results of the phenol compound-linked pentagalloyl glucose derivative according to an aspect (GBD-CP1-1.0) (Formula 39); FIG. 3A shows HPLC results, and FIG. 3B shows HRMS (SEI) results.

FIG. 4 shows results of confirming an anti-cancer effect when the phenol compound-linked pentagalloyl glucose derivative compound according to an aspect (GBD-CP1-1.0) (Formula 39) is treated to colon cancer cells; FIG. 4A shows results of confirming an effect of inhibiting colorectal cancer cell growth according to the concentration and time, and FIG. 4B shows results of confirming a morphological change of colorectal cancer cells according to the concentration and time.

FIG. 5 shows results of confirming an effect of inhibiting colorectal cancer cell growth when the phenol compound-linked pentagalloyl glucose derivative according to an aspect (GBD-CP1-1.0) (Formula 39) is treated to colorectal cancer cells; FIG. 5A confirms an inhibitory effect on anchorage-independent growth of colorectal cancer cells in soft-agar, and FIG. 5B shows results of confirming average numbers of colony-forming cells for the result of FIG. 5A.

FIG. 6 shows results of confirming an anti-cancer effect after treating the phenol compound-linked pentagalloyl glucose derivative according to an aspect (GBD-CP1-1.0) (Formula 39) to oral squamous cell carcinoma cells; FIG. 6A shows results of confirming an growth inhibitory effect on oral squamous cell carcinoma cells according to the concentration and time, FIG. 6B shows results of confirming an inhibitory effect on anchorage-independent growth of oral squamous cell carcinoma cells in soft-agar, and FIG. 6C shows results of confirming average numbers of colony-forming cells for the result of FIG. 6B.

FIG. 7 shows results of confirming an effect of lowering blood sugar after treating the phenol compound-linked pentagalloyl glucose derivative (GBD-CP1-1.0) (Formula 39) to mouse models with diabetes induced by treating STZ; FIG. 7A shows results of measuring changes in the weight of the mice, and FIG. 7B shows results of measuring degrees of decrease in the blood glucose levels of the mice.

### MODE OF DISCLOSURE

Hereinafter, the present disclosure will be described in more detail through examples. However, these examples are intended to illustrate at least one embodiment, and the scope of the present disclosure is not limited to these examples.

### Examples Preparation of Phenol Compound-Linked Pentagalloyl Glucose Derivatives

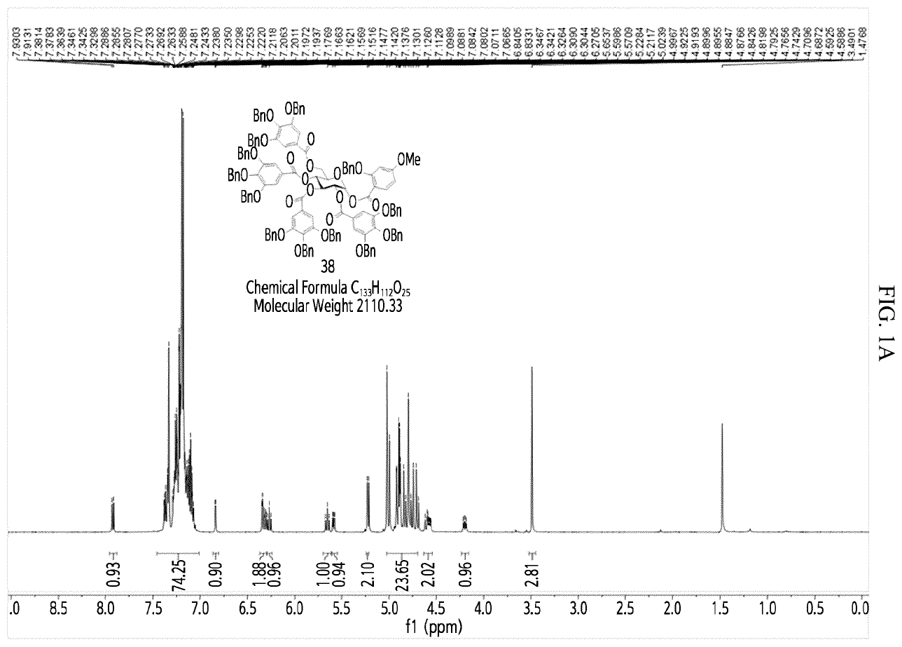

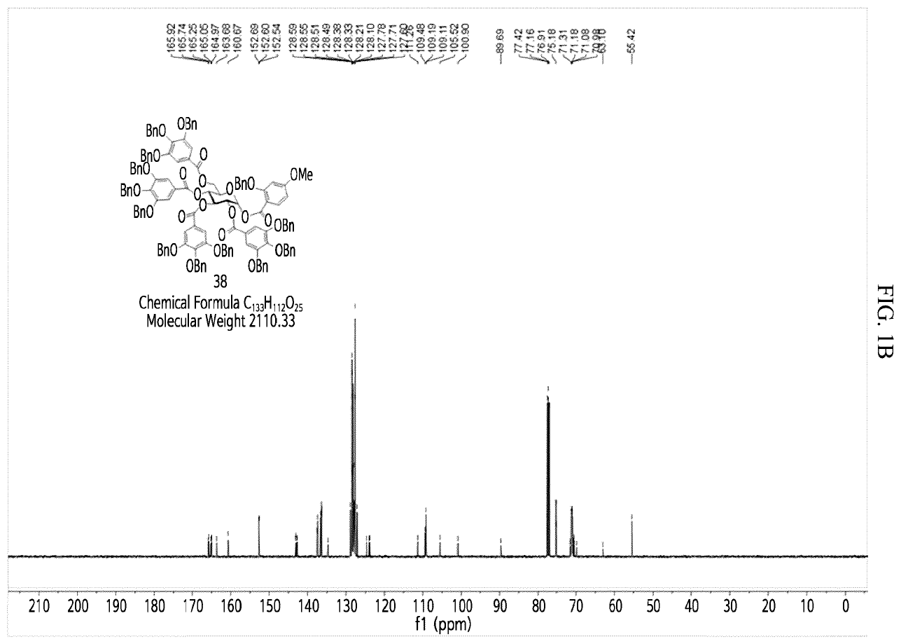

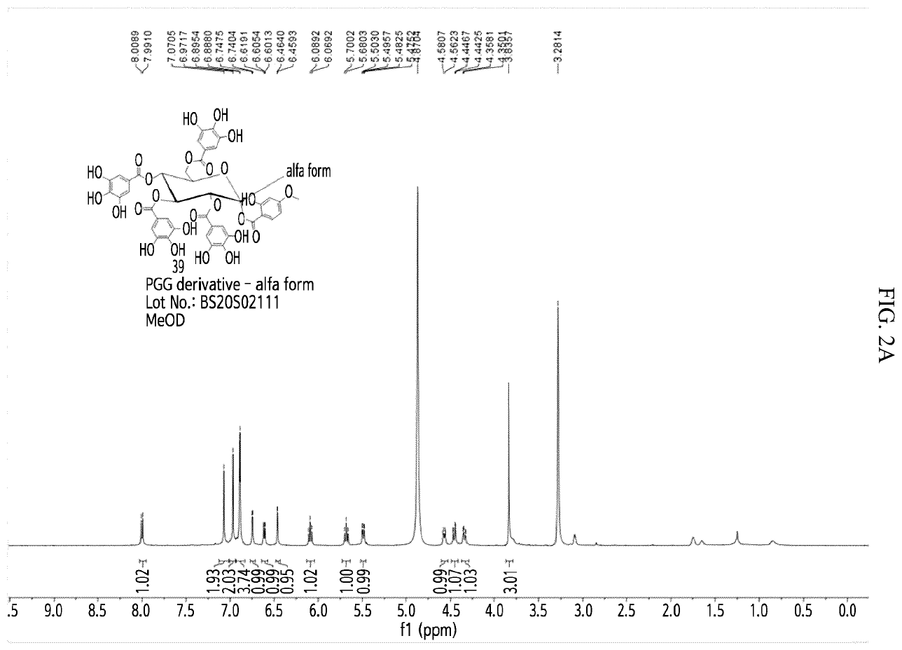

Phenol compound-linked pentagalloyl glucose derivatives were prepared through a two-process reaction. First, (3R,4S,5R,6R)-2-hydroxy-6-(((3,4,5-tris(benzyloxy)benzoyl)oxy)methyl)tetrahydro-2H-pyran-3,4,5-triyltris(3,4,5-tris(benzyloxy)benzoate) (Formula 36, 940 mg, 0.5 mmol) was dissolved in 24 mL of methylene chloride (DCM), and 2-(benzyloxy)-4-methoxybenzoic acid (Formula 37, 180 mg, 0.7 mol), EDCI (134 mg, 0.7 mmol), and DMAP (86 mg, 0.7 mmol) were dissolved. Then, N,N-Diisopropylethylamine (DIPEA, 218 μL, 1.25 mmol) was slowly added at room temperature and mixed. The mixture obtained by mixing was stirred at room temperature for 12 hours, and the reaction was monitored by using the thin-layer chromatography (TLC) method. 120 mL of methylene chloride (DCM) was added to the reaction mixture after the monitoring was completed, and the sample was washed 3 times with 30 mL of brine. After washing, the organic layer was separated, dried by using anhydrous Na₂SO₄, filtered and evaporated, and the obtained material was purified by using a column to obtain a mixture of an α form and β form (α:β=2.5:1) (Formula 37). The obtained mixture was purified again by using a column ((toluene:EtOAc=100:1.5 to 100:2), and a pure compound of the α form ((2R,3R,4S,5R,6R)-2-((2-(benzyloxy)-4-methoxybenzoyl)oxy)-6-(((3,4,5-tris(benzyloxy)benzoyl)oxy)methyl)tetrahydro-2H-pyran-3,4,5-triyltris(3,4,5-tris(benzyloxy)benzoate) (Formula 38, 400 mg, yield: 36%) was obtained. [¹H NMR (500 MHZ, Chloroform-d) δ7.92 (d, J=8.6 Hz, 1H), 7.44-7.02 (m, 74H), 6.84 (d, J=3.7 Hz, 1H), 6.37-6.30 (m, 2H), 6.27 (t, J=10.0 Hz, 1H), 5.65 (t, J=10.0 Hz, 1H), 5.58 (dd, J=10.2, 3.7 Hz, 1H), 5.23 (d, J=1.7 Hz, 2H), 5.07-4.67 (m, 23H), 4.63-4.52 (m, 2H), 4.20 (dd, J=12.0, 5.0 Hz, 1H), 3.49 (s, 3H). ¹³C NMR (126 MHZ, Chloroform-d) δ165.9, 165.7, 165.2, 165.0, 165.0, 163.7, 160.7, 152.7, 152.7, 152.6, 152.5, 143.2, 143.1, 142.9, 142.6, 137.6, 137.5, 137.4, 136.8, 136.5, 136.4, 136.4, 134.7, 128.9, 128.6, 128.6, 128.5, 128.5, 128.4, 128.4, 128.3, 128.2, 128.2 128.1, 128.1, 128.1, 127.9, 127.8, 127.7, 127.6, 127.2, 124.7, 124.1, 123.9, 123.9, 111.3, 109.5, 109.2, 109.1, 105.5, 100.9, 89.7, 75.2, 75.2, 75.2, 71.6, 71.3, 71.2, 71.1, 71.0, 70.8, 70.6, 70.5, 69.9, 63.1, 55.4.]. ¹H NMR results of Formula 38 are shown in FIG. 1A, and ¹³C NMR results are shown in FIG. 1B.

The reaction is shown in Scheme 1 below:

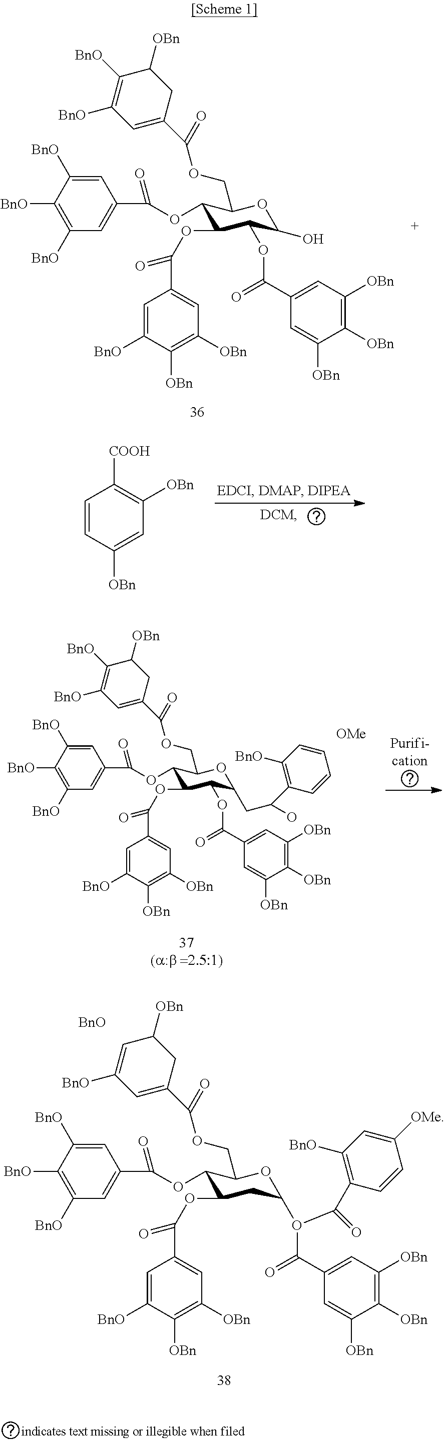

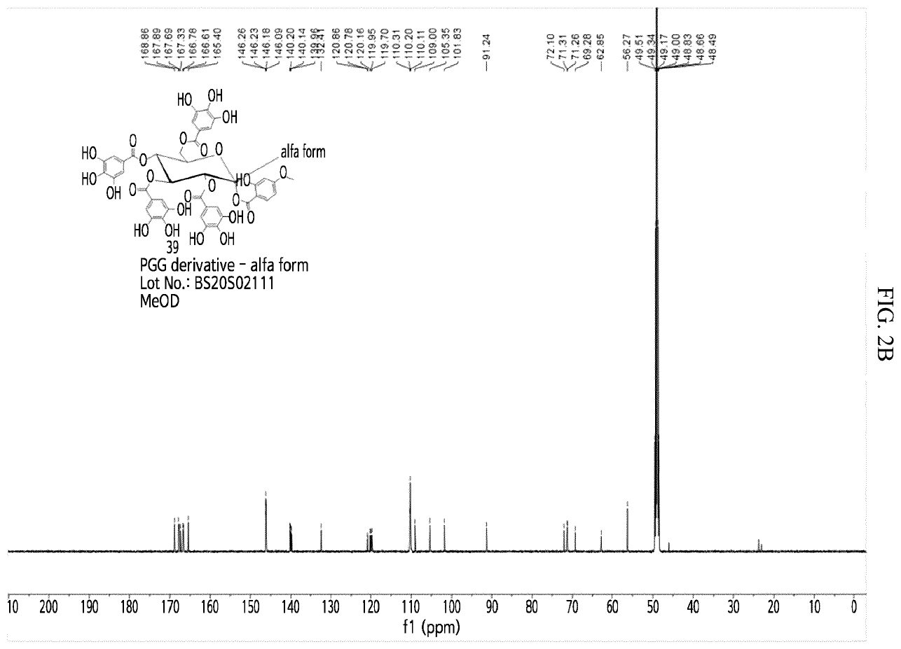

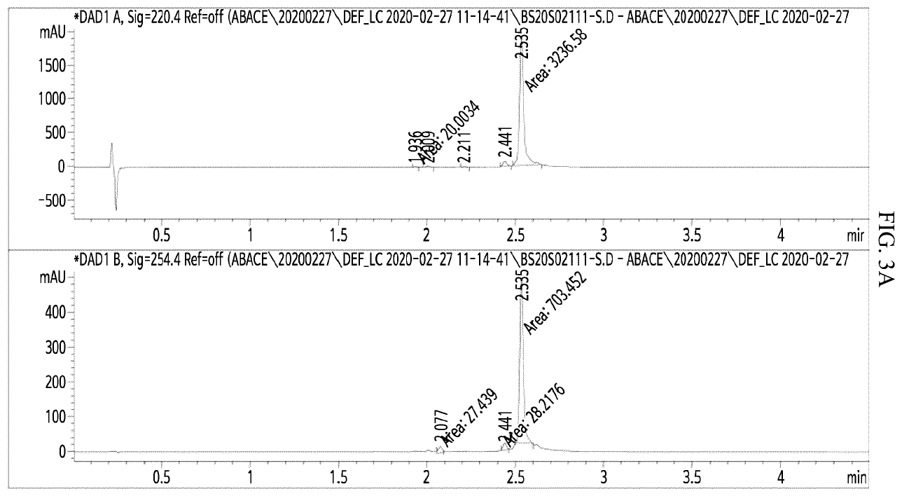

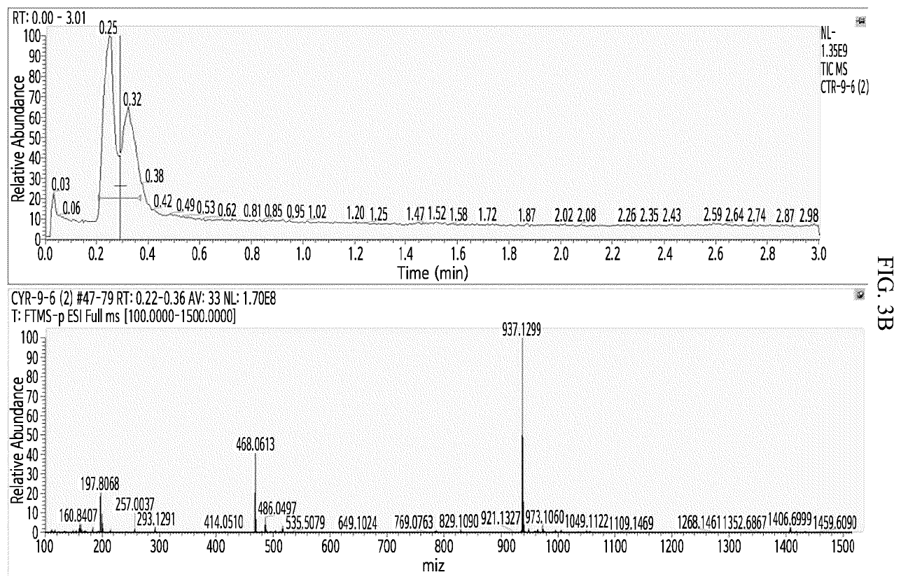

Next, in order to prepare a PGG derivative-α form from the α-form compound (Formula 38) prepared according to Scheme 1, (2R,3R,4S,5R,6R)-2-((2-(benzyloxy)-4-methoxybenzoyl)oxy)-6-(((3,4,5-tris(benzyloxy)benzoyl)oxy)methyl)tetrahydro-2H-pyran-3,4,5-triyl tris(3,4,5-tris(benzyloxy)benzoate) (Formula 38, 400 mg) was dissolved in 30 ml of methanol at room temperature, and 10% Pd/C (600 mg) was slowly added and mixed under a hydrogen gas atmosphere (pressure 1 atm). The mixed mixture was heated to 40° C. and stirred for 12 hours to 16 hours (overnight), and the reaction process was monitored by the NMR method. After the monitoring was completed, the reaction mixture was filtered by using a celite plug and washed with methanol. The solvent used was removed by concentrating the sample in vacuum to obtain a brown solid target compound PGG derivative-α form (Formula 39, 151 mg, purity, 94.6% at 220 nm, 92.7% at 254 nm, yield, 85%). and the same was named as GBD-CP1-1.0. [¹H NMR (500 MHZ, Methanol-d4) δ8.01 (d, J=9.0 Hz, 1H), 7.07 (s, 2H), 6.97 (s, 2H), 6.89 (d, J=3.7 Hz, 4H), 6.74 (d, J=3.5 Hz, 1H), 6.61 (dd, J=8.9, 2.4 Hz, 1H), 6.46 (d, J=2.4 Hz, 1H), 6.09 (t, J=10.0 Hz, 1H), 5.68 (t, J=9.9 Hz, 1H), 5.49 (dd, J=10.3, 3.6 Hz, 1H), 4.57 (dd, J=10.1, 2.8 Hz, 1H), 4.46 (dd, J=12.7, 2.2 Hz, 1H), 4.34 (dd, J=12.6, 4.0 Hz, 1H), 3.84 (s, 3H). ¹³C NMR (126 MHZ, Methanol-d4) δ168.9, 167.9, 167.7, 167.3, 166.8, 166.6, 165.4, 146.3, 146.2, 146.2, 146.1, 140.2, 140.2, 140.0, 139.8, 132.4, 120.9, 120.8, 120.2, 120.0, 119.7, 110.3, 110.2, 110.1, 109.0, 105.4, 101.8, 91.2, 72.1, 71.3, 71.3, 69.3, 62.9, 56.3. HRMS (ESI): [M-H]-calculated for C42H33025-: 937.1316; found: 937.1299]. ¹H NMR results of Formula 39 are shown in FIG. 2A, and ¹³C NMR results are shown in FIG. 2B. In addition, the HPLC results of Formula 39 are shown in FIG. 3A, and the HRMS (SEI) results are shown in FIG. 3B.

The reaction is shown in Scheme 2 below:

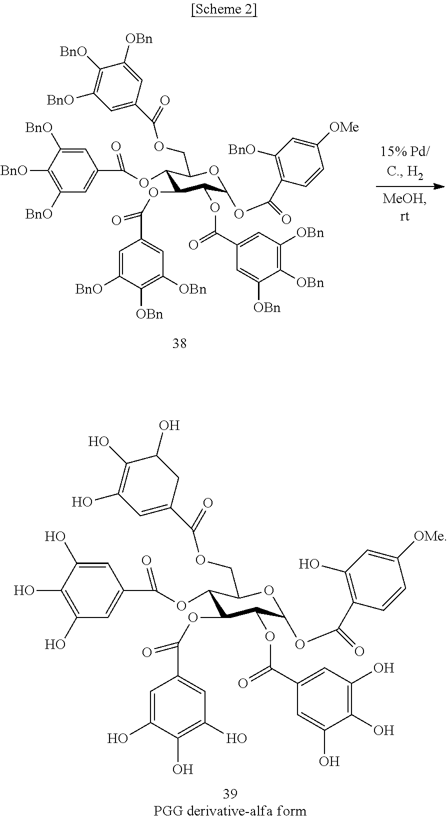

### Experimental Example 1. Experimental Preparation and Method for Confirming Activity of Phenol Compound-Linked Pentagalloyl Glucose Derivative

### 1-1. Cell Culture

Colon cancer cells HCT116 were cultured in McCoy's 5A medium containing 10% FBS, 100 U/mL penicillin, and 100 μg/mL streptomycin, and oral squamous cell carcinoma cells Ca9-22 were cultured in Dulbecco Modified Eagle Medium (DMEM) medium containing 10% FBS, 100 U/mL penicillin, 100 μg/mL streptomycin, 0.03% glutamine, and 1 mM sodium pyruvate, each at 37° C. and under 5% CO₂ conditions.

### 1-2. Cell Proliferation Assay

HCT116 and Ca9-22 cancer cells were each seeded in a 96-well plate at 1×10³ cells/well (100 μL), and stabilized, and then the derivative compound (GBD-CP1-1.0) dissolved in DMSO was treated at 5 μM, 10 μM, and 20 μM, and then the cells were cultured at 37° C., under the condition of 5% CO₂. Each cancer cell line was cultured for 24 hours, 48 hours, 72 hours, and 92 hours, and then the cells were taken at each time period, 10 μL of Cell Counting Kit-8 (CCK-8) solution was added to each well, and incubated at 37° C. for 1 hour, then absorbance values were measured by using a microplate reader at 450 nm.

### 1-3. Soft-agar Assay

0.3% top agar mixtures (1 mL) including 8×10³ cells/mL of HCT116 or Ca9-22 cells, and respectively including 5 μM, 10 M, and 20 μM of the derivative compound (GBF-CP1-1.0) were applied to the upper layer of 1% bottom agar mix (3 mL) respectively including 5 M, 10 μM and 20 μM of the derivative compound (GBD-CP1-1.0) prepared in a 6-well plate, and the samples were incubated at 37° C., under 5% CO₂ conditions for 7 days, and then, the numbers of colonies on each plate were counted. In the top agar and bottom agar mixtures, for HCT116, McCoy's 5A medium containing 10% FBS, and 50 μg/mL gentamicin was used, and for Ca9-22, DMEM medium containing 10% FBS, 50 μg/mL gentamicin, 0.03% glutamine, and 1 mM sodium pyruvate was used.

### 1-4. STZ-Induced Diabetic Mouse Model (STZ-Induced Diabetic Animal Model)

After acclimatizing 5-week-old male C57BL/6N mice to the laboratory environment for 1 week, streptozotocin (STZ) was dissolved in 0.1 M of citrate buffer (pH 4.5) and injected intraperitoneally once at a dose of 150 mg/kg to induce diabetes. Each experimental group was divided into a control (0.1 M citrate buffer treatment), STZ treatment group, STZ+insulin treatment group, and STZ+GBD-CP1-1.0 treatment group (n=10). When the test groups were treated with STZ, and the blood glucose level reached 400 mg/dl or higher, insulin and the derivative compound (GBD-CP1-1.0) were injected intraperitoneally by 5 IU each, and blood glucose levels in the tail arterial blood of the mice in each experimental group were measured at an interval of 1 hour, 2 hours, 4 hours, 6 hours, 12 hours, and 24 hours. Body weights of the mice in each experimental group were measured by using a simple scale before administration of the reagent.

### Experimental Example 2. Confirmation of Treatment Effect of Phenol Compound-Linked Pentagalloyl Glucose Derivative for Colorectal Cancer

Colorectal cancer cells (HCT116 cells) cultured by the method of Experimental Example 1-1 for varying time (24 hr, 48 hr, 72 hr, and 96 hr) were treated with the derivative compound (GBD-CP1-1.0) prepared by the above example with varying concentrations (0 μM, 5 μM, 10 μM, and 20 μM), and the growth inhibitory effect on colorectal cancer cells and morphological changes were confirmed.

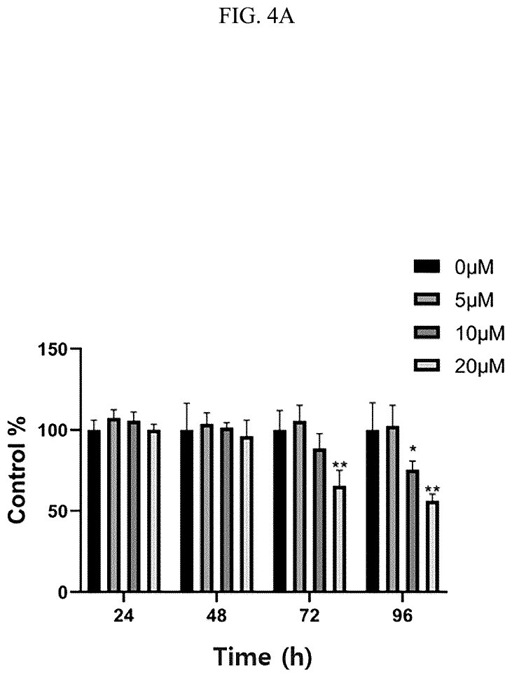

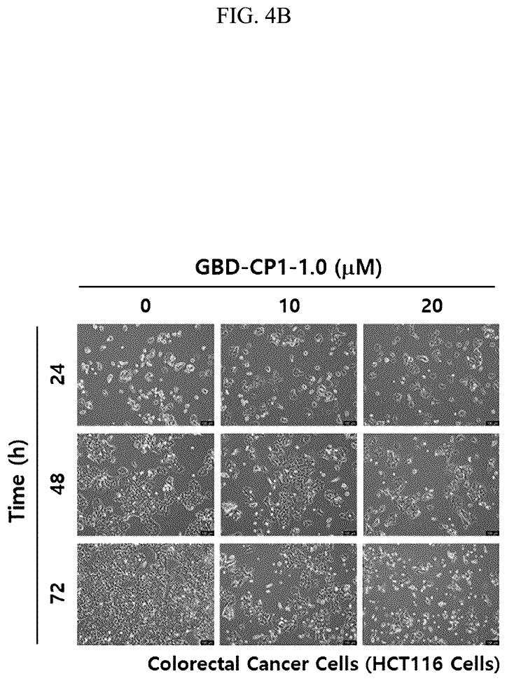

As a result, as shown in FIGS. 4A and 4B, when the derivative compound was administered at a concentration of 10 μM and 20 μM, growth of the colorectal cancer cells was inhibited after 72 hours. In addition, in the case of morphological changes of the cells, it was confirmed that when the derivative compound was treated at a concentration of 10 μM and 20 μM, proliferation of the colorectal cancer cells was inhibited compared to the control group after 48 hours to 72 hours (*, p<0.05; **, p<0.001).

In addition, degrees of inhibition of anchorage-independent growth for colorectal cancer cells were confirmed in soft-agar by varying the concentration (0 μM, 5 μM, 10 μM, and 20 μM) of the derivative compound prepared in the example.

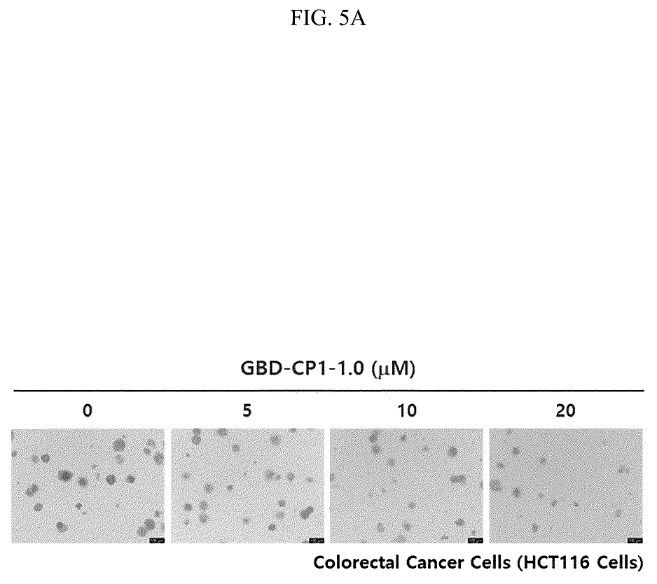

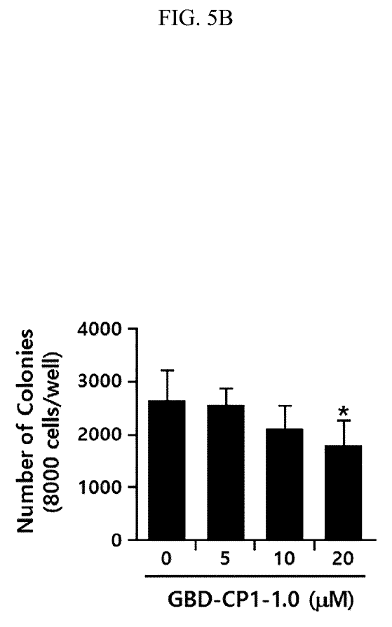

As a result, as shown in FIG. 5A, it was confirmed that anchorage-independent growth of the colorectal cancer cells was inhibited as the concentration of the derivative compound increased, and as a result of confirming inhibition of anchorage-independent growth with average numbers of colony-forming cells, as shown in FIG. 5B, it was confirmed that the number of colonies decreased by the derivative compound as the concentration of the derivative compound increased (*, p<0.05; **, p<0.001).

### Experimental Example 3. Confirmation of Treatment Effect of Phenol Compound-Linked Pentagalloyl Glucose Derivative for Oral Squamous Cell Carcinoma

Oral squamous cell carcinoma cells (Ca9-22 cells) cultured by the method of Experimental Example 1-1 for varying time (24 hr, 48 hr, 72 hr, and 96 hr) were treated with the derivative compound (GBD-CP1-1.0) prepared by the example with varying concentrations (0 μM, 5 μM, 10 μM, and 20 μM), and the growth inhibitory effect on oral squamous cell carcinoma cells and morphological changes were confirmed.

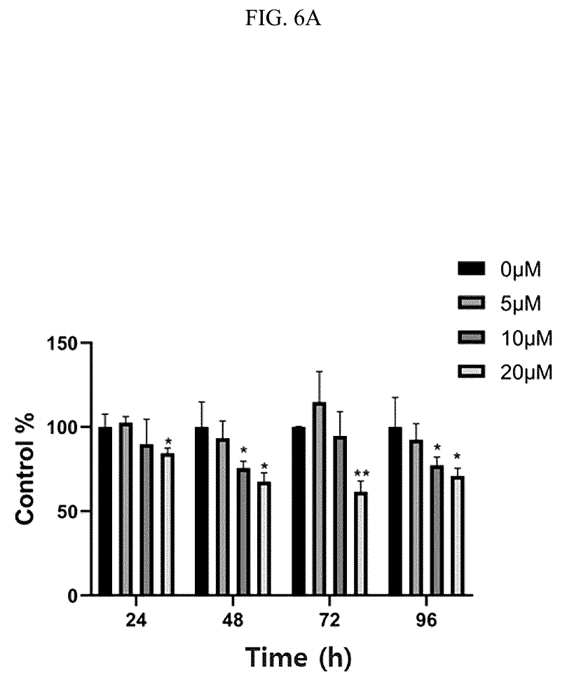

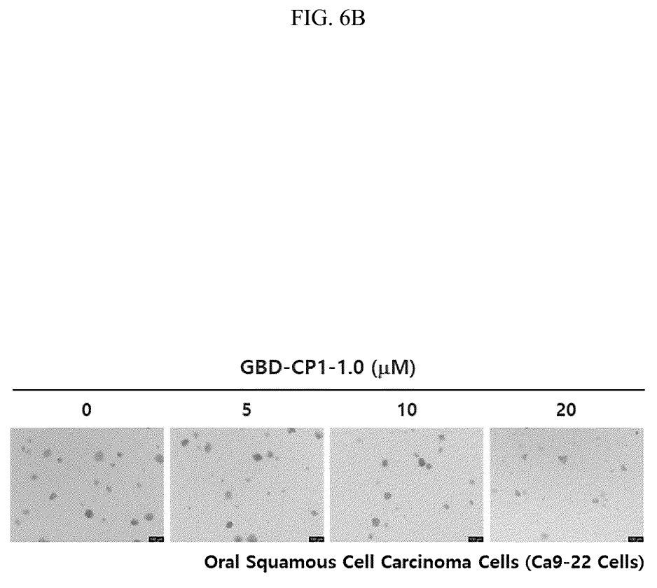

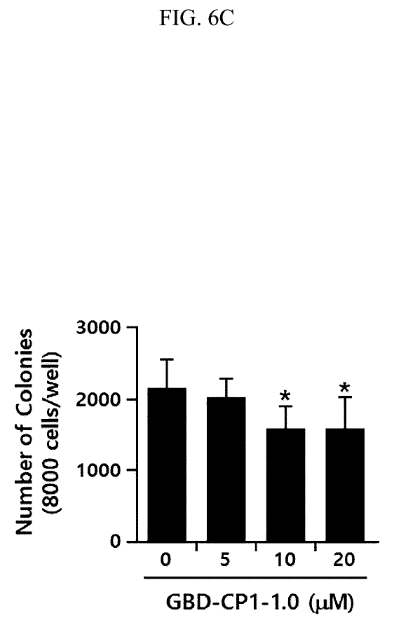

As a result, as shown in FIG. 6A, when the derivative compound was administered at a concentration of 10 μM and 20 μM, the growth of oral squamous cell carcinoma cells was inhibited after 24 hours (*, <0.05; **, p<0.001).

In addition, degrees of inhibition of anchorage-independent growth for oral squamous cell carcinoma cells were confirmed in soft-agar by varying the concentration (0 μM, 5 μM, 10 μM, and 20 μM) of the derivative compound prepared in the example.

As a result, as shown in FIG. 6B, it was confirmed that anchorage-independent growth of the squamous cell carcinoma cells was inhibited by the derivative compound as the concentration of the derivative compound increased, and as a result of confirming inhibition of anchorage-independent growth with average numbers of colony-forming cells, as shown in FIG. 6C, it was confirmed that the number of colonies decreased when the derivative compound was treated at a concentration of 10 μM and 20 μM (*, p<0.05; **, p<0.001).

### Experimental Example 4. Confirmation of Anti-Diabetic Effect of Phenol Compound-Linked Pentagalloyl Glucose Derivative in STZ-Induced Diabetic Mouse Model

In the same manner as in Experimental Example 1-4, mice with diabetes induced by administrating STZ were treated with the derivative compound prepared in Example 1, and weight changes and blood glucose lowering efficacy in the mice of each experimental group were confirmed.

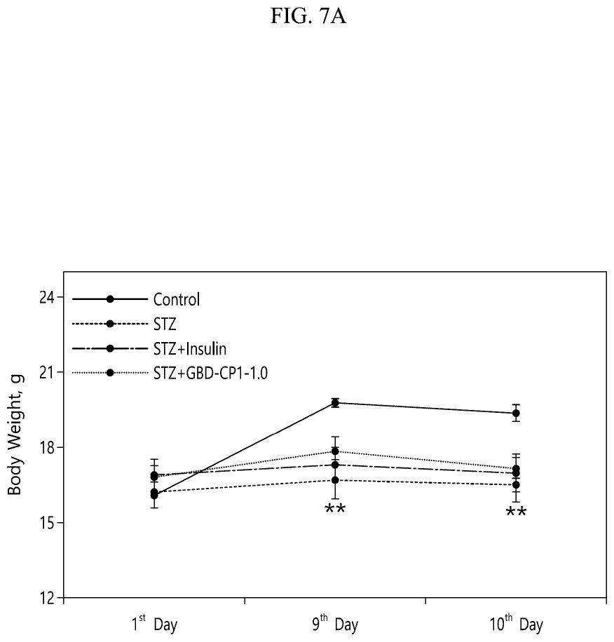

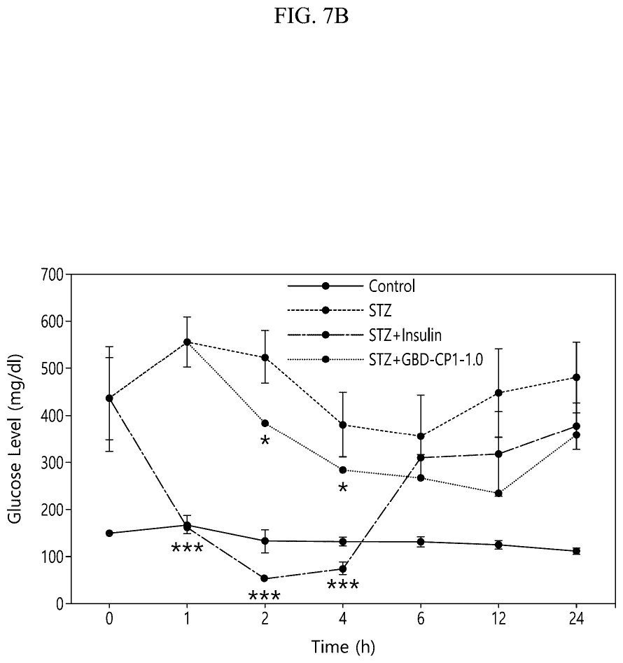

As a result, it was confirmed that the weights of the experimental group mice (STZ+GBD-CP1-1.0 treatment) were increased compared to that of the control group mice (STZ treatment), as shown in FIG. 7A. In addition, as shown in FIG. 7B, it was confirmed that levels of glucose in the blood decreased over time in the experimental group mice (STZ+GBD-CP1-1.0 treatment) compared to that of the control group mice (STZ treatment).

1-18. (canceled)

19. A compound represented by Formula 1 or a pharmaceutically acceptable salt thereof: 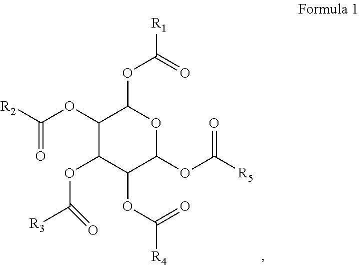
wherein in Formula 1, R₁, R₂, R₃, R₄ or R₅ is each independently a compound of Formula 2 or 3: 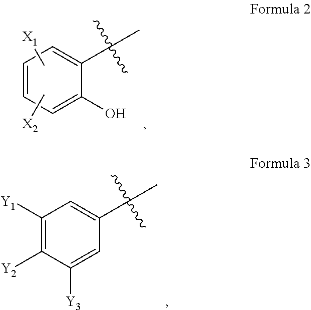
at least one of R₁, R₂, R₃, R₄, and R₅ is a compound represented by Formula 2,
in Formula 2, X₁ or X₂ is each independently H, OH, a C₁ to C₁₀ alkyl that is substituted or unsubstituted, a C₂ to C₁₀ alkenyl that is substituted or unsubstituted, a C₂ to C₁₀ alkynyl that is substituted or unsubstituted, or a C₁ to C₁₀ alkoxy that is substituted or unsubstituted, and
in Formula 3, Y₁, Y₂, or Y₃ is each independently H, OH, a C₁ to C₁₀ alkyl that is substituted or unsubstituted, a C₂ to C₁₀ alkenyl that is substituted or unsubstituted, a C₂ to C₁₀ alkynyl that is substituted or unsubstituted, or a C₁ to C₁₀ alkoxy that is substituted or unsubstituted.

20. The compound or pharmaceutically acceptable salt thereof of claim 19, wherein in Formula 2, X₁ or X₂ is each independently H, OH, or a C₁ to C₁₀ alkyl that is substituted or unsubstituted.

21. The compound or pharmaceutically acceptable salt thereof of claim 19, wherein in Formula 3, Y₁, Y₂, or Y₃ is each independently H, OH, or a C₁ to C₁₀ alkyl that is substituted or unsubstituted.

22. The compound or pharmaceutically acceptable salt thereof of claim 19, wherein the compound of Formula 2 is a compound represented by Formula 4 below:

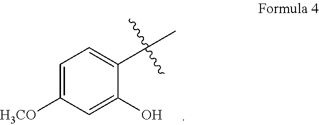 [SMILES: *c1ccc(OC)cc1O]

23. The compound or pharmaceutically acceptable salt thereof of claim 19, wherein the compound of Formula 3 is a compound represented by Formula 5 below:

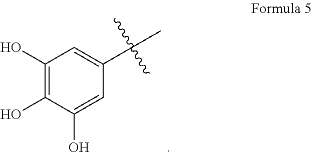 [SMILES: *c1cc(O)c(O)c(O)c1]

24. A pharmaceutical composition comprising a compound represented by Formula 1 or a pharmaceutically acceptable salt thereof: 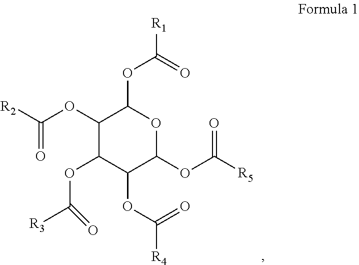
wherein in Formula 1, R₁, R₂, R₃, R₄, or R₅ is each independently a compound of Formula 2 or 3: 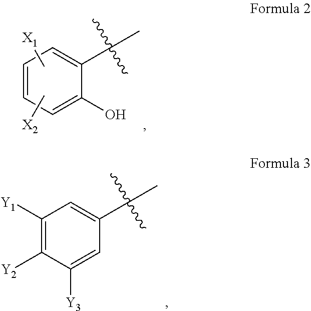
at least one of R₁, R₂, R₃, R₄, and R₅ is a compound represented by Formula 2,
in Formula 2, X₁ or X₂ is each independently H, OH, a C₁ to C₁₀ alkyl that is substituted or unsubstituted, a C₂ to C₁₀ alkenyl that is substituted or unsubstituted, a C₂ to C₁₀ alkynyl that is substituted or unsubstituted, or a C₁ to C₁₀ alkoxy that is substituted or unsubstituted; and
in Formula 3, Y₁, Y₂, or Y₃ is each independently H, OH, a C₁ to C₁₀ alkyl that is substituted or unsubstituted, a C₂ to C₁₀ alkenyl that is substituted or unsubstituted, a C₂ to C₁₀ alkynyl that is substituted or unsubstituted, or a C₁ to C₁₀ alkoxy that is substituted or unsubstituted.

25. The pharmaceutical composition of claim 24, for preventing or treating cancer, diabetes, cancer for diabetic patients or diabetes for cancer patients.

26. The pharmaceutical composition of claim 25, wherein the cancer is a blood cancer or a solid cancer.

27. The pharmaceutical composition of claim 26, wherein the blood cancer is at least one selected from the group consisting of acute myeloid leukemia, acute lymphoblastic leukemia, chronic myelogenous leukemia, multiple myeloma, and lymphoma.

28. The pharmaceutical composition of claim 26, wherein the solid cancer is at least one selected from the group consisting of breast cancer, colorectal cancer, head and neck cancer, lung cancer, stomach cancer, skin cancer, colon cancer, prostate cancer, bladder cancer, kidney cancer, rectal cancer, thyroid cancer, liver cancer, cervical cancer, skin cancer, anal cancer, urethral cancer, ovarian cancer, esophageal cancer, and pancreatic cancer.

29. The pharmaceutical composition of claim 24, wherein in Formula 2, X₁ or X₂ is each independently H, OH, or a C₁ to C₁₀ alkyl that is substituted or unsubstituted.

30. The pharmaceutical composition of claim 24, wherein in Formula 3, Y₁, Y₂, or Y₃ is each independently H, OH, or a C₁ to C₁₀ alkyl that is substituted or unsubstituted.

31. The pharmaceutical composition of claim 24, wherein the compound of Formula 2 is a compound represented by Formula 4 below:

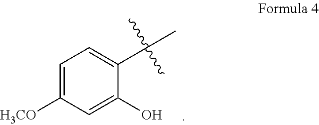 [SMILES: *c1ccc(OC)cc1O]

32. The pharmaceutical composition of claim 24, wherein the compound of Formula 3 is a compound represented by Formula 5 below:

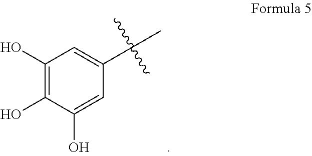 [SMILES: *c1cc(O)c(O)c(O)c1]

33. A method of preventing or treating cancer, diabetes, cancer for diabetic patients or diabetes for cancer patients, comprising administering a compound represented by Formula 1 or a pharmaceutically acceptable salt thereof to a subject in need thereof: 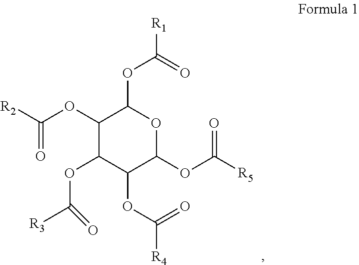
wherein in Formula 1, R₁, R₂, R₃, R₄ or R₅ is each independently a compound of Formula 2 or 3: 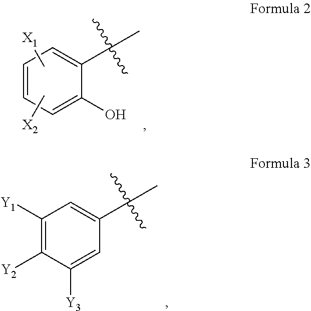
at least one of R₁, R₂, R₃, R₄, and R₅ is a compound represented by Formula 2,
in Formula 2, X₁ or X₂ is each independently H, OH, a C₁ to C₁₀ alkyl that is substituted or unsubstituted, a C₂ to C₁₀ alkenyl that is substituted or unsubstituted, a C₂ to C₁₀ alkynyl that is substituted or unsubstituted, or a C₁ to C₁₀ alkoxy that is substituted or unsubstituted; and
in Formula 3, Y₁, Y₂, or Y₃ is each independently H, OH, a C₁ to C₁₀ alkyl that is substituted or unsubstituted, a C₂ to C₁₀ alkenyl that is substituted or unsubstituted, a C₂ to C₁₀ alkynyl that is substituted or unsubstituted, or a C₁ to C₁₀ alkoxy that is substituted or unsubstituted.

34. The method of claim 33, wherein in Formula 2, X₁ or X₂ is each independently H, OH, or a C₁ to C₁₀ alkyl that is substituted or unsubstituted.

35. The method of claim 33, wherein in Formula 3, Y₁, Y₂, or Y₃ is each independently H, OH, or a C₁ to C₁₀ alkyl that is substituted or unsubstituted.

36. The method of claim 33, wherein the compound of Formula 2 is a compound represented by Formula 4 below:

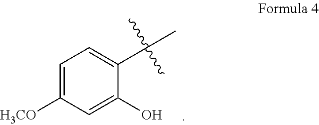 [SMILES: *c1ccc(OC)cc1O]

37. The method of claim 33, wherein the compound of Formula 3 is a compound represented by Formula 5 below: 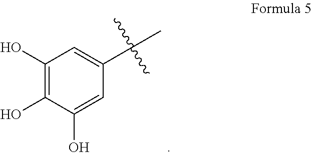 [SMILES: *c1cc(O)c(O)c(O)c1]
